(19) United States
(12) Patent Application Publication
(10) Pub. No.: US 2020/0357495 A1
(43) Pub. Date: Nov. 12, 2020

(54) METHOD, SERVER, AND PROGRAM FOR PROVIDING HEALTHCARE DATA

(71) Applicant: SONGAREE IT CO., LTD., Seoul (KR)

(72) Inventor: Dae Jin KIM, Seongnam-si (KR)

(73) Assignee: SONGAREE IT CO., LTD., Seoul (KR)

(21) Appl. No.: 16/940,526

(22) Filed: Jul. 28, 2020

Related U.S. Application Data
(63) Continuation of application No. PCT/KR2019/003277, filed on Mar. 21, 2019.

(30) Foreign Application Priority Data
Mar. 22, 2018 (KR) 10-2018-0033023

(51) Int. Cl. G16H 10/60; G16H 40/20; G16H 40/67

(52) U.S. Cl. CPC G16H 10/60; G16H 40/67; G16H 40/20

(57) ABSTRACT

The present invention relates to a healthcare data providing method for allowing viewing of personal health data stored in a user application, according to a user's approval, so as to enable a medical staff to check personal health data of the user before or during treatment of the user.

### CROSS-REFERENCE TO RELATED APPLICATIONS

The present application is a continuation of International Patent Application No. PCT/KR2019/003277, filed on Mar. 21, 2019, which is based upon and claims the benefit of priority to Korean Patent Application No. 10-2018-0033023, filed on Mar. 22, 2018. The disclosures of the above-listed applications are hereby incorporated by reference herein in their entirety.

### BACKGROUND

Embodiments of the inventive concept described herein relate to a server for providing healthcare data to manage the health of a user, a method therefor, and a program therefor.

Recently, with the development of medical technology, as there has been an increase in interest in health, the Internet of things (IoT) and the medical technology are frequently combined with each other.

In general, when a patient visits a hospital, he or she directly visit the hospital and checks in at the hospital to be treated or makes an appointment with the doctor over his or her telephone or the Internet to be treated.

However, although a patient makes an appointment with the doctor, he or she simply makes an appointment and the doctor does not check records of the patient in advance. Thus, efficient patient management is not performed.

Thus, when the doctor starts to treat the patient, he or she asks the patient questions about a health state, previous treatment experiences, or symptoms of the patient and prescribes medication to the patient depending on answers. Thus, it may take a long time and information obtained from the patient may be uncertain.

A technology serving as the background of the inventive concept is disclosed in Korean Patent Application Laying-open No. 10-2001-0097151 or the like, but, it does not propose a fundamental solution to the above-mentioned problems.

### SUMMARY

Embodiments of the inventive concept provide a server for providing healthcare data to permit a medical staff client to view personal health data (e.g., a personal health record) stored in a user application such that the medical staff client may check the personal health data (e.g., the personal health record) of a user before or while treating the user, a method therefor, and a program therefor.

Embodiments of the inventive concept provide a server for providing healthcare data to display a warning message by means of a user application such that medical staff may inquire about related data, when the user application monitors information about oxygen saturation or a pulse of a child and discovers an abnormal symptom of the child, a method therefor, and a program therefor.

The technical problems to be solved by the inventive concept are not limited to the aforementioned problems, and any other technical problems not mentioned herein will be clearly understood from the following description by those skilled in the art to which the inventive concept pertains.

According to an exemplary embodiment, a method for providing healthcare data may include receiving, by a server, a request to view personal health data of a specific user from a medical staff client, the personal health data including health information or medical information and being stored in a user application, permitting, by the server, the medical staff client to view the personal health data as the user approves to view the personal health data, and ending the permission to view the personal health data, as a certain time elapses or as medical staff store chart data. The health information may include at least one of body information, a family history, or a social history of the user. The medical information may include at least one of symptom information about a symptom of the user or information obtained through hospital treatment. The chart data may be stored by adding medical information depending on operations of the medical staff after the personal health data of the user is viewed by the medical staff client.

Furthermore, the permitting to view the personal health data may include transmitting, by the server, a message requesting to view the personal health data to the user application and receiving, by the server, an approval time for viewing and a time limit for viewing from the user application and permitting, by the server, the medical staff client to view the personal health data. The ending of the permission to view the personal health data may include ending the permission to view the personal health data as the time limit for viewing elapses or as the medical staff store the chart data.

Furthermore, the permitting to view the personal health data may include receiving, by the server, the request to view the personal health data and a viewing authentication number from the medical staff client, matching, by the server, the received viewing authentication number with a viewing authentication number stored in the user application, and permitting, by the server, the medical staff client to view the personal health data, when the viewing authentication numbers are identical to each other according to the matched result.

Furthermore, the method may further include transmitting, by the server, a message requesting to extend a viewing time to the user application, when receiving the message from the medical staff client and additionally permitting, by the server, the medical staff to view the personal health data by a time input from the user application.

Furthermore, the method may further include receiving, by the server, the symptom information about the symptom of the user from the user application and receiving, by the server, a request to recommend a hospital according to the symptom information and analyzing, by the server, the symptom information to select a department and recommending, by the server, one or more hospitals according to the department to the user application.

Furthermore, the method may further include inquiring, by the server, about a specialty of a specific medical staff client, when receiving a request to view personal health data of the user from the specific medical staff client, determining, by the server, that the user is in an emergency state, when the specialty corresponds to an emergency department, and permitting, by the server, the specific medical staff client to view the personal health data.

Furthermore, the permitting to view the personal health data may further include permitting, by the server, the medical staff client to view personal health data of the family of the user or personal health data of a child of the user, as the user approves to view the personal health data. The personal health data of the family of the user may be personal health data of a family member who is approved to share the personal health data with the user. The personal health data of the child of the user may be personal health data of a child who is registered with a user application account and may include measurement data being received from a sensor worn on a body of the child and being stored in the user application.

According to an exemplary embodiment, a server for providing healthcare data may include a reception unit that receives a request to view personal health data of a specific user from a medical staff client and a viewing permission unit that permits the medical staff client to view the personal health data as the user approves to view the personal health data. The personal health data may include health information or medical information and is stored in a user application. The health information may include at least one of body information, a family history, or a social history of a patient. The medical information may include at least one of symptom information about a symptom of the user or information obtained through hospital treatment. The chart data may be stored by adding medical information depending on operations of medical staff after the personal health data of the user is viewed by the medical staff client.

Furthermore, the user application may receive and store personal health data including health information or medical information of a child of the user, may receive and monitor measurement data for oxygen saturation and a pulse of the child of the user from a sensor worn or added to a body of the child, may display a warning message when an abnormal symptom of the child is discovered, and may store the measurement data and the abnormal symptom of the child in the personal health data of the child.

Furthermore, the reception unit may receive a request to view personal health data of a child of the specific user from the medical staff client. The viewing permission unit may permit the medical staff client to view the personal health data of the child as the user approves to view the personal health data and may end the viewing as a certain time elapses or as the medical staff store chart data.

In addition, another method for implementing the inventive concept, another system therefor, and a computer-readable storage medium storing a computer program for executing the method may be further provided.

According to an exemplary embodiment, a program for providing healthcare data may be combined with a computer which is hardware and may be stored in a medium to execute the method for providing the healthcare data.

### BRIEF DESCRIPTION OF THE FIGURES

The above and other objects and features will become apparent from the following description with reference to the following figures, wherein like reference numerals refer to like parts throughout the various figures unless otherwise specified, and wherein:

FIG. 1 is a drawing schematically illustrating a server for providing healthcare data according to an embodiment of the inventive concept;

FIG. 2 is a signal sequence diagram illustrating a method for providing healthcare data according to an embodiment of the inventive concept;

FIG. 3 is a drawing illustrating an example where a family doctor views health information and medical information of a user according to an embodiment of the inventive concept;

FIG. 4 is a drawing illustrating an example where a server provides a response method according to a symptom of a user according to an embodiment of the inventive concept;

FIG. 5 is a block diagram illustrating a server for providing healthcare data according to an embodiment of the inventive concept;

FIG. 6 is a drawing illustrating an example where a user application monitors an abnormal symptom of a child according to an embodiment of the inventive concept;

FIG. 7 is a drawing illustrating an example of inputting a profile of a child of a user according to an embodiment of the inventive concept;

FIGS. 8 and 9 are drawings illustrating an example of providing information about a growth record of a child of a user according to an embodiment of the inventive concept;

FIG. 10 is a drawing illustrating an example of providing information about vaccination of a child according to an embodiment of the inventive concept; and

FIG. 11 is a drawing illustrating an example where a user application displays a medicine taking notification message according to another embodiment of the inventive concept.

### DETAILED DESCRIPTION

Advantages, features, and methods of accomplishing the same will become apparent with reference to embodiments described in detail below together with the accompanying drawings. However, the inventive concept is not limited by embodiments disclosed hereinafter, and may be implemented in various forms. Rather, these embodiments are provided to so that this disclosure will be through and complete and will fully convey the concept of the invention to those skilled in the art, and the inventive concept will only be defined by the appended claims.

Terms used in the specification are used to describe embodiments of the inventive concept and are not intended to limit the scope of the inventive concept. In the specification, the terms of a singular form may include plural forms unless otherwise specified. The expressions “comprise” and/or “comprising” used herein indicate existence of one or more other elements other than stated elements but do not exclude presence of additional elements. Like reference numerals designate like elements throughout the specification, and the term “and/or” may include each of stated elements and one or more combinations of the stated elements. The terms such as “first” and “second” are used to describe various elements, but it is obvious that such elements are not restricted to the above terms. The above terms are used only to distinguish one element from the other. Thus, it is obvious that a first element described hereinafter may be a second element within the technical scope of the inventive concept.

Unless otherwise defined herein, all terms (including technical and scientific terms) used in the specification may have the same meaning that is generally understood by a person skilled in the art. Also, terms which are defined in a dictionary and commonly used should be interpreted as not in an idealized or overly formal detect unless expressly so defined.

Hereinafter, an embodiment of the inventive concept will be described in detail with reference to the accompanying drawings.

Prior to description, meanings of terms used in the specification will be described in brief. However, because the description of terms is to help understand the specification, it should be noted that the description of terms is not used as the meaning of limiting the technical scope of the inventive concept unless explicitly described as limiting the inventive concept.

Medical staff refer to a doctor who proceeds with treating a user or a medical assistant, such as a nurse, who has a partnership with a corresponding doctor.

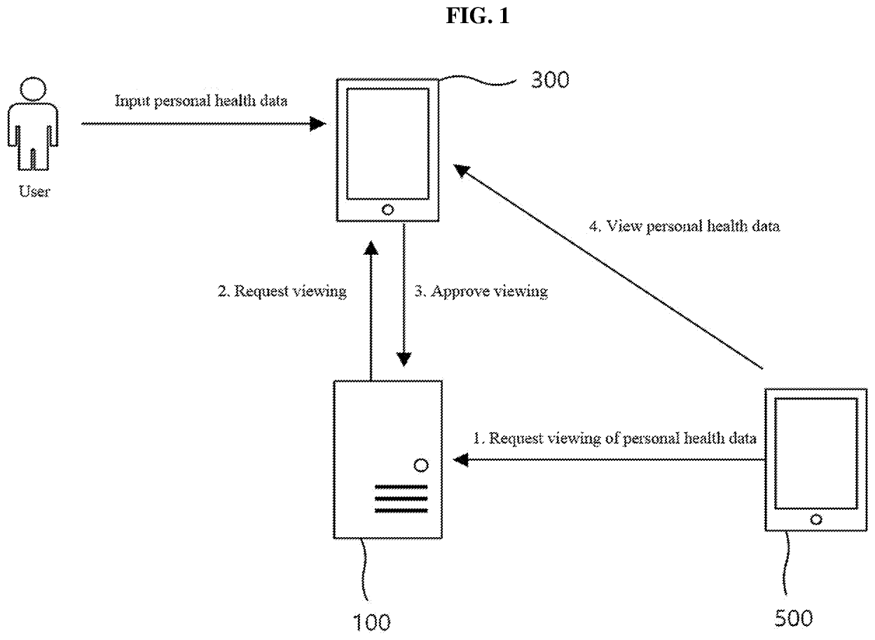

FIG. 1 is a drawing schematically illustrating a server for providing healthcare data according to an embodiment of the inventive concept.

An embodiment of the inventive concept will be described in brief with reference to FIG. 1.

A user application 300 may receive and store personal health data (e.g., a personal health record) from a user.

In this case, the personal health data may include health information and medical information of the user. The health information may include at least one of body information, a family history, or a social history of the user. The medical information may include at least one of symptom information about a symptom of the user or information obtained through hospital treatment.

A server 100 may receive a request to view personal health data, which is for verifying the personal health data of the user before or while treating the user, from a medical staff client 500, may transmit the request to the user application 300, and may receive approval to view the personal health data.

The server 100 may permit the medical staff client 500 to access the user application 300 and view the personal health data of the user.

Thus, the server 100 does not store any information about the personal health data of the user by acting as a go-between between the user application 300 and the medical staff client 500. The server 100 may allow the user to safely store the personal health data and may allow medical staff to check the personal health data of the user to be helped to treat the user by acting as a go-between between the user application 300 and the medical staff client 500.

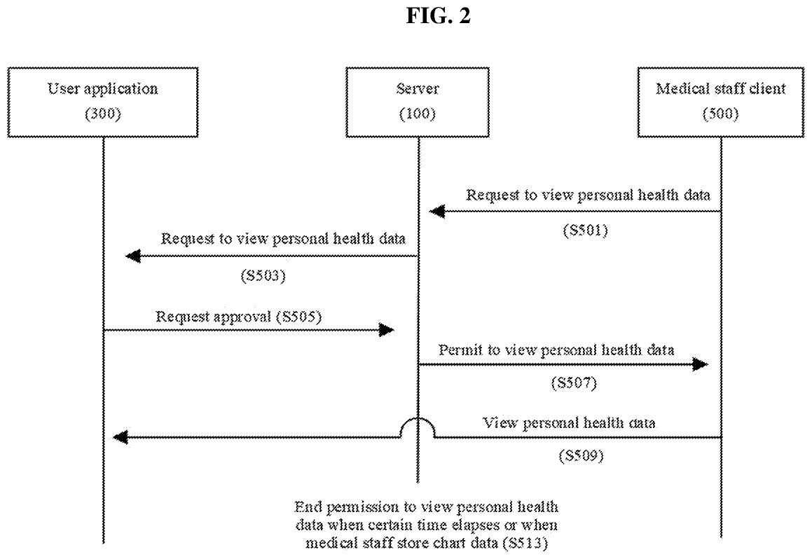

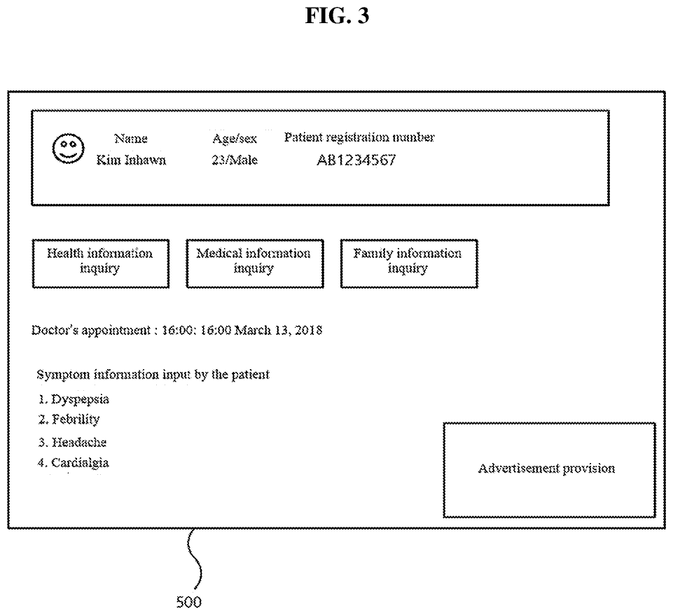

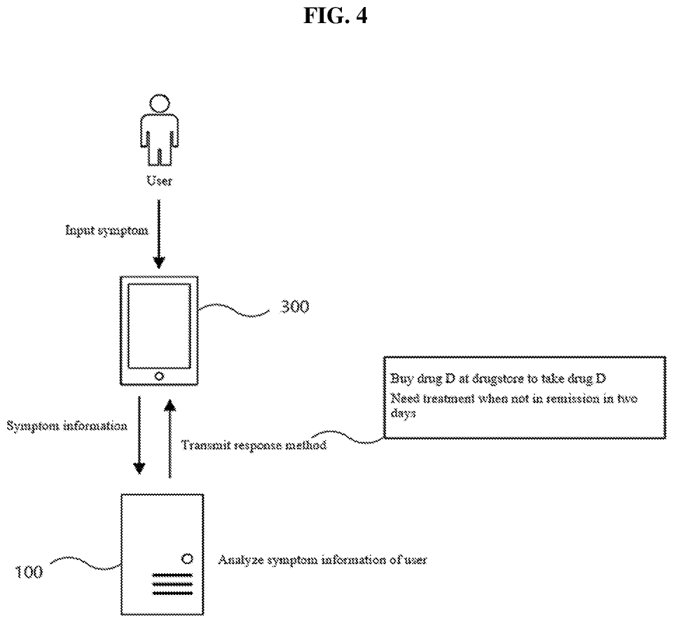

FIG. 2 is a signal sequence diagram illustrating a method for providing healthcare data according to an embodiment of the inventive concept. FIG. 3 is a drawing illustrating an example where a family doctor views health information and medical information of a user according to an embodiment of the inventive concept. FIG. 4 is a drawing illustrating an example where a server provides a response method according to a symptom of a user according to an embodiment of the inventive concept.

A description will be given of the method for providing the healthcare data according to an embodiment of the inventive concept with reference to FIG. 2.

First of all, in operation S501, a server 100 may receive a request to view personal health data of a specific user from a medical staff client 500.

In detail, the receiving of the request to view the personal health data of the user from the medical staff client 500 may be performed before or while medical staff treat a user. That medical staff request personal health data for treatment of the user is applicable at any time.

Furthermore, a method for inquiring of the user may use a name, a gender, an age, a patient registration number, or the like of the user.

Subsequent to operation S501, in operation S503, the server 100 may transmit a message requesting to view the personal health data to the user application 300.

In detail, the server 100 may display information of the medical staff client 500 who requests to view the personal health data by means of the user application 300, such that the user identifies the medical staff client 500. For example, information such as a hospital name, doctor names, and departments of medical staff may be displayed.

Subsequent to operation S503, in operation S505, the server 100 may receive approval to view the personal health data from the user application 300. In operation S507, the server 100 may permit the medical staff client 500 to view the personal health data.

In operation S509, the medical staff client 500 may view the personal health data stored in the user application 300.

Thus, the medical staff client 500 may view the personal health data of the user as shown in FIG. 3.

In detail, the personal health data may include health information and medical information of the user. The medical staff client 500 may inquire about family information of the user. In this case, the family information of the user may include information such as a family history for determining health of the user.

Furthermore, the server 100 may provide an advertisement at one side of a screen for inquiring about the personal health data.

In this case, the server 100 may inquire about departments of the medical staff client 500 and may provide an advertisement associated with the inquired department. Preferably, this means that the server 100 provides an advertisement of a medical device, a pharmaceutical company, or the like of a corresponding specialty depending on the specialty of a doctor, such as the internal medicine department, the dental department, or the surgery department.

In operation S513, the server 100 may end the permission to view the personal health data, as a certain time elapses or as medical staff store chart data.

In this case, the chart data may refer to being stored by adding medical information according to operations of medical staff after the medical staff client 500 views the personal health data stored in the user application 300.

In detail, the chart data may mean that the server 100 ends the permission to view the personal health data when a certain time elapses or when medical staff store the chart result. The chart data may refer to medical records and chart records of the user and may mean that medical staff store medical records and chart records of the user by means of the medical staff client 500 after treating the user.

As another embodiment, the permitting (S507) to view the personal health data may include receiving, by the server 100, an approval time for viewing and a time limit for viewing from the user application 300 and permitting, by the server 100, the medical staff client 500 to view the personal health data.

The ending (S513) of the permission to view the personal health data may include ending, by the server 100, the permission to view the personal health data as the time limit for viewing elapses or as medical staff store chart data.

This means that the user approves the request to view the personal health data by means of the user application 300 and inputs a time limit for viewing together to permit to view the personal health data during only the time.

As another embodiment, the permitting (S507) to view the personal health data may include receiving, by the server 100, a request to view personal health data and a viewing authentication number from the medical staff client 500, matching, by the server 100, the viewing authentication number received from the medical staff client 500 with a viewing authentication number stored in the user application 300, and permitting the medical staff client 500 to view the personal health data when the viewing authentication numbers are identical to each other according to the matched result.

This is a configuration for allowing the user to set the viewing authentication number in the user application 300 to view the personal health data of the user and allow only the medical staff client 500, which knows the viewing authentication number, to view the personal health data of the user to doubly maintain security.

Subsequent to the ending (S513) of the permission to view the personal health data, the method may further include, when receiving a message requesting to extend a viewing time from the medical staff client 500, transmitting, by the server 100, the message to the user application 300 and additionally permitting, by the server 100, the medical staff client 500 to view the personal health data by a time input from the user application 300.

This is an operation of being permitted to additionally view the personal health data from the user, when medical staff do not check the personal health data of the user within the permitted viewing time due to a schedule or the other problems. This additionally permits the medical staff client 500 to view the personal health data by the time input from the user application 300, such that the medical staff client may check the personal health data.

Referring to FIG. 4, as another embodiment, the method may further include receiving, by the server 100, symptom information about a symptom of the user from the user application 300 and receiving, by the server 100, a request to recommend a hospital according to the symptom information, and analyzing, by the server 100, the received symptom information to select a department and recommending, by the server 100, one or more hospitals according to the department to the user application 300.

In detail, the server 100 may analyze the symptom information of the user and may transmit a response method.

The server 100 may recommend the one or more hospitals according to the department to the user application 300, which may ascertain a location of the user by means of the user application 300 and may recommend a hospital around the user.

As another embodiment, the method may further include, when receiving a request to view personal health data of the user from the specific medical staff client 500, inquiring about a department of the medical staff client 500 and determining, by the server 100, whether the user is in an emergency state when the department corresponds to an emergency department and permitting, by the server 100, the medical staff client 500 to view the personal health data.

For example, when the user is rushed to the emergency room due to an accident and when medical staff who take charge of treating the user in the emergency room wants to check information such as chronic disease of the user, this may mean that the server 100 permits the medical staff client 500 to view personal health data of the user.

As another embodiment, the permitting (S507) to view the personal health data may further include permitting, by the server 100, the medical staff client 500 to view personal health data of the family of the user or personal health data of a child of the user.

In this case, the personal health data of the family of the user may be personal health data of a family member who is approved to share the personal health data with the user, and the personal health data of the child of the user may be personal health data of a child who is registered with a user application account, which includes measurement data which is received from a sensor worn on a body of the user and is stored in a user application.

In detail, the user application may store the personal health data of the child of the user, including health information or medical information of the child, and may receive and store measurement data for oxygen saturation and a pulse of the child of the user from a sensor worn on the body of the child.

Furthermore, the user application may monitor measurement data. When an abnormal symptom of the child is discovered, the user application may display a warning message and may store a degree or time of the abnormal symptom.

Furthermore, the family member refers to a father, a mother, a spouse, or the like of the user. It is preferable that authority is granted through a user client when sharing between the user and the family member is approved after they access the server using their applications respectively installed in their terminals.

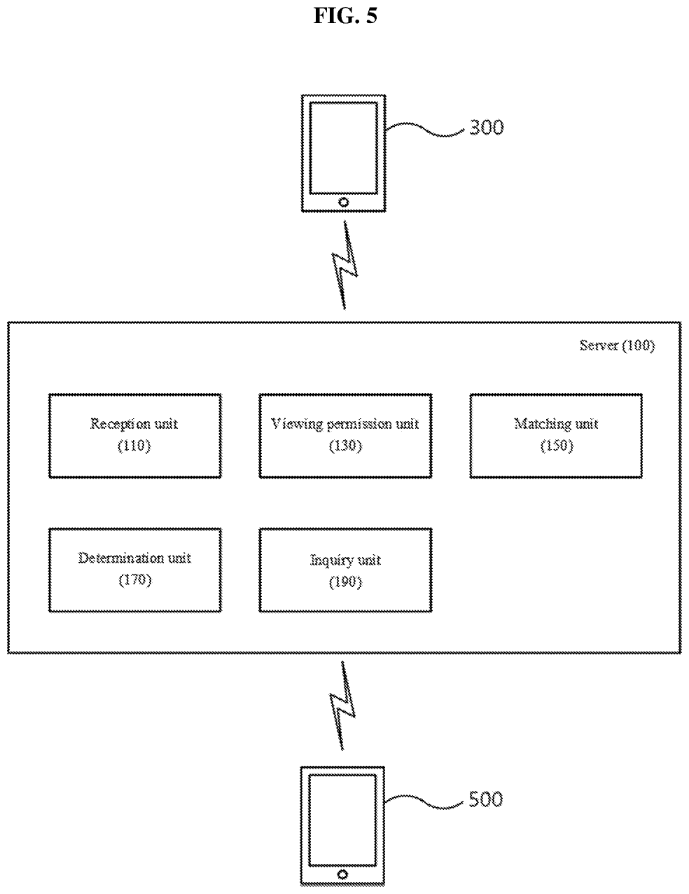

FIG. 5 is a block diagram illustrating a server for providing healthcare data according to an embodiment of the inventive concept.

A description will be given of a server 100 for providing healthcare data according to an embodiment of the inventive concept with reference to FIG. 5.

The server 100 may include a reception unit 110, a viewing permission unit 130, a matching unit 150, a determination unit 170, and an inquiry unit 190.

The reception unit 110 may receive a request to view personal health data (e.g., a personal health record) of a specific user from a medical staff client 500.

In this case, the personal health data (e.g., the personal health record) may include health information or medical information of the user, which may be stored in a user application 300.

The health information may include at least one of body information, a family history, or a social history of the user. The medical information may include at least one of symptom information about a symptom of the user or information obtained through hospital treatment.

The viewing permission unit 130 may permit a medical staff client 500 to view the personal health data as the user approves to view the personal health data and may end the viewing as a certain time elapses or as media staff store chart data.

The chart data may refer to being stored by adding medical information according to operations of medical staff after the medical staff client 500 views the personal health data stored in the user application 300.

In detail, the chart data may refer to medical records and chart records of the user and may mean that medical staff store medical records and chart records of the user by means of the medical staff client 500 after treating the user.

As another embodiment, the viewing permission unit 130 may receive an approval time for viewing and a time limit for viewing from the user application 300, may permit the medical staff client 500 to view the personal health data of the user, and may end the permission to view the personal health data as the time limit for viewing elapses or as medical staff store chart data.

As another embodiment, the reception unit 110 of the server 100 may receive a request to view personal health data and a viewing authentication number from the medical staff client 500. The matching unit 150 may match the viewing authentication number received by the reception unit 110 of the server 100 with a viewing authentication number stored in the user application 300, and may permit the medical staff client 500 to view the personal health data of the user when the viewing authentication numbers are identical to each other according to the matched result.

Furthermore, when receiving a message requesting to extend a viewing time from the medical staff client 500, the reception unit 110 of the server 100 may transmit the message to the user application 300. The viewing permission unit 130 may additionally permit the medical staff client 500 to view the personal health data by a time input from the user application 300.

Furthermore, the server 100 may receive symptom information about a symptom of the user from the user application 300 and may receive a request to recommend a hospital according to the symptom information.

The determination unit 170 of the server 100 may analyze the symptom information received from the user application 300 to select a department and may recommend one or more hospitals according to the department to the user application 300.

When the reception unit 110 of the server 100 receives a request to view personal health data of the user from the specific medical staff client 500, the inquiry unit 190 may inquire about a department of the specific medical staff client 500, may determine whether the user is in an emergency state when the department corresponds to an emergency department, and may permit the specific medical staff client 500 to view personal health data.

For example, the inquiry unit 190 may access information of the medical staff client 500 and may inquire about a hospital name and a specialty of the medical staff client 500.

Furthermore, the user application 300 may receive and store the personal health data including health information or medical information of a child of the user.

The user application 300 may receive and monitor measurement data for oxygen saturation and a pulse of the child of the user from a sensor 600 worn or attached to a body of the child of the user. When an abnormal symptom of the child is discovered, the user application 300 may display a warning message and may store the measurement data and the abnormal symptom of the child in the personal health data of the child.

Furthermore, the reception unit 110 of the server 100 may receive a request to view personal health data of a child of a specific user from the medical staff client 500. The viewing permission unit 130 may permit the medical staff client 500 to view the personal health data of the child as the user approves to view the personal health data and may end the viewing as a certain time elapses or as medical staff store chart data.

Thus, the medical staff may check measurement data for oxygen saturation and a pulse of the child and a time when an abnormal symptom occurs and may treat the child.

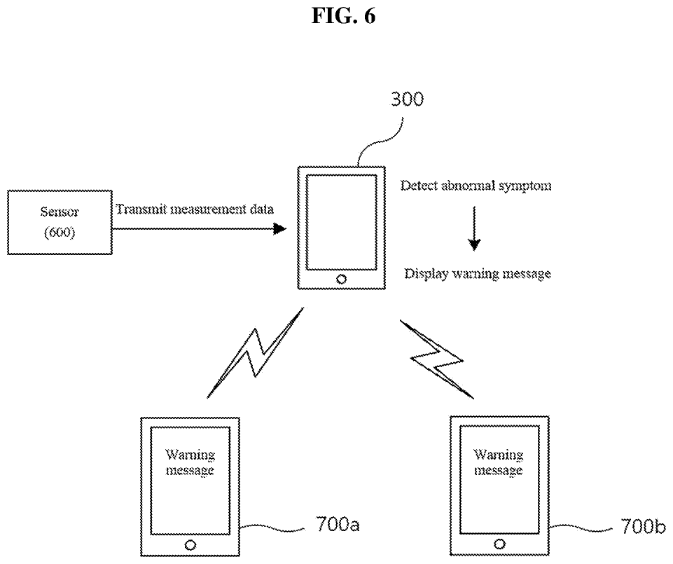

FIG. 6 is a drawing illustrating an example where a user application monitors an abnormal symptom of a child according to an embodiment of the inventive concept.

Referring to FIG. 6, when a user application 300 receives one or more other uses as additional protectors and when a user does not check a warning message during a predetermined time, the user application 300 may display the warning message by means of an application 700a (hereinafter referred to as “another user application”) installed in a terminal of another user.

For example, a mother of the child may not know when an abnormal symptom of the child will occur 24 hours a day and may not check a warning message of the user application 300 because of putting her smartphone down for a while to do other works. Thus, the user application 300 may register another user such as a father of the child as an additional protector and may display the warning message by means of other user applications 700a and 700b to cope with the situation.

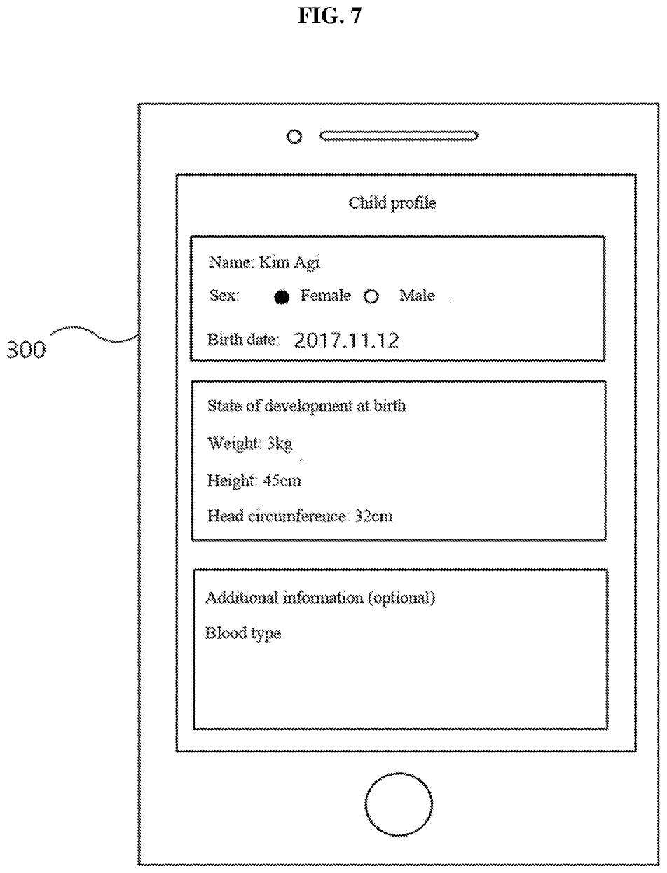

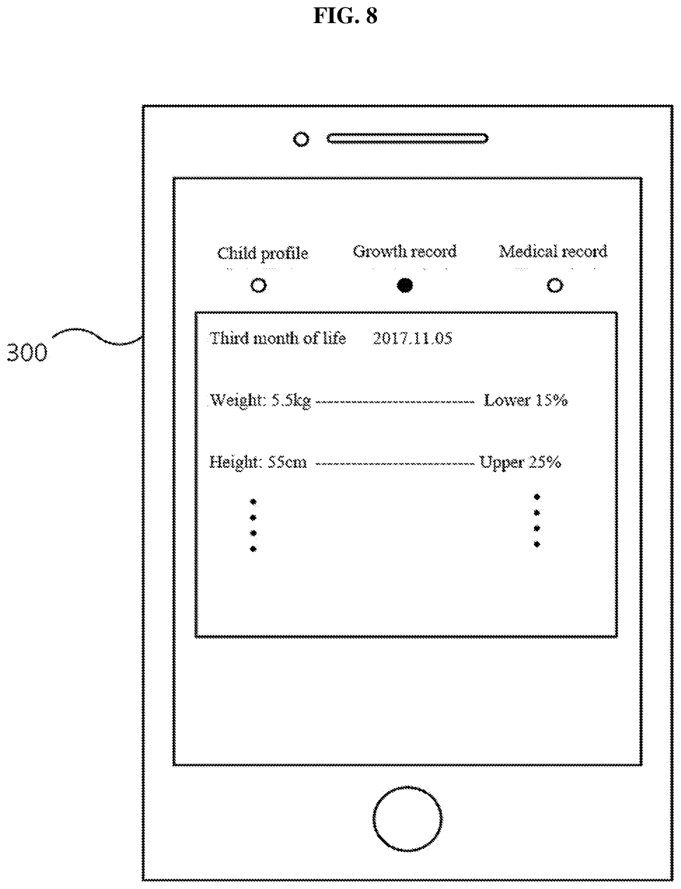

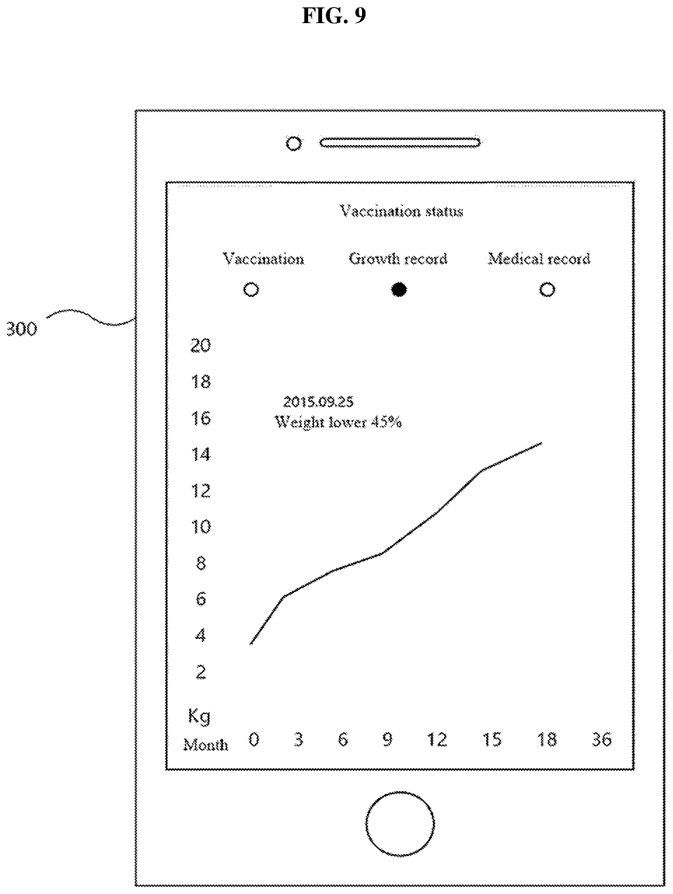

FIG. 7 is a drawing illustrating an example of inputting a profile of a child of a user according to an embodiment of the inventive concept. FIGS. 8 and 9 are drawings illustrating an example of providing information about a growth record of a child of a user according to an embodiment of the inventive concept.

Referring to FIGS. 7 to 9, a user application 300 may receive and store body information of the child of the user at a certain period and may provide growth information of the child as a graph image using the received body information, which may provide average growth information of children of the same age together.

Thus, the user may check information about the growth record of the child and may visit a hospital with the child to allow the child to be treated, when a growth state of the child is less than an average value.

A sensor 600 and the user application 300 may be connected in a wireless fidelity (Wi-Fi) or ultra wide band (UWB) mode. When communication between the sensor 600 and the user application 300 is disconnected, a warning message may be displayed by means of the user application 300 and other user applications 700a and 700b.

In this case, although the sensor 600 and the user application 300 are a little distant from each other, because they are frequently disconnected in a Bluetooth mode and because they are frequently interrupted by other signals in the Bluetooth mode, it is preferable that the sensor 600 and the user application 300 use the Wi-Fi or UWB mode for stably transmitting and receiving signals without communication distance constraints.

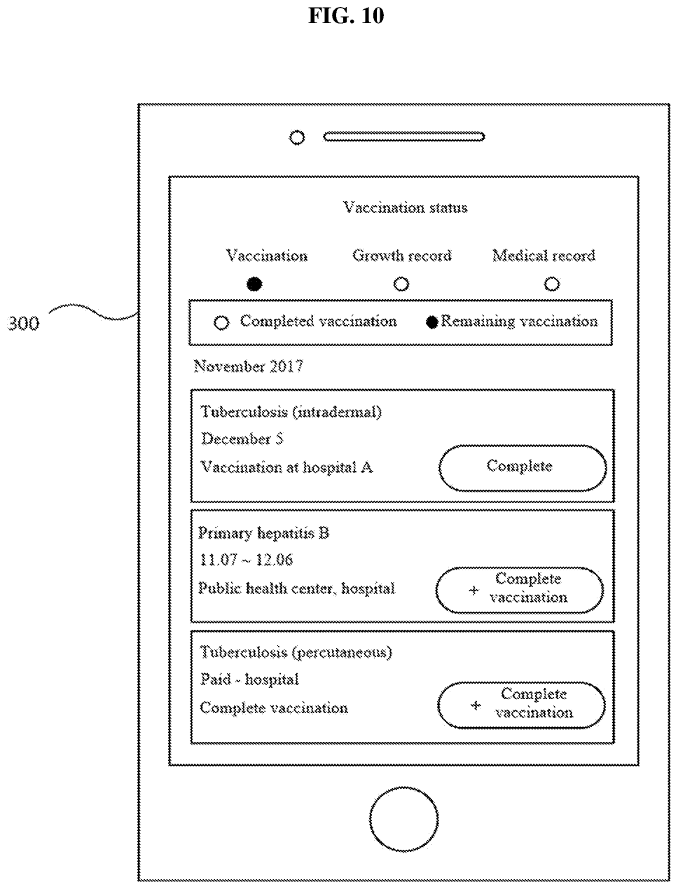

FIG. 10 is a drawing illustrating an example of providing information about vaccination of a child according to an embodiment of the inventive concept.

Referring to FIG. 10, a user application 300 may provide vaccination information a child of a user needs and information about a vaccination period using a profile of the child of the user. When it is period for the child to be vaccinated, the user application 300 may display a notification message.

Furthermore, the user application 300 may periodically download vaccination information from a server 100 and may additionally store a vaccination list added to an existing vaccination list.

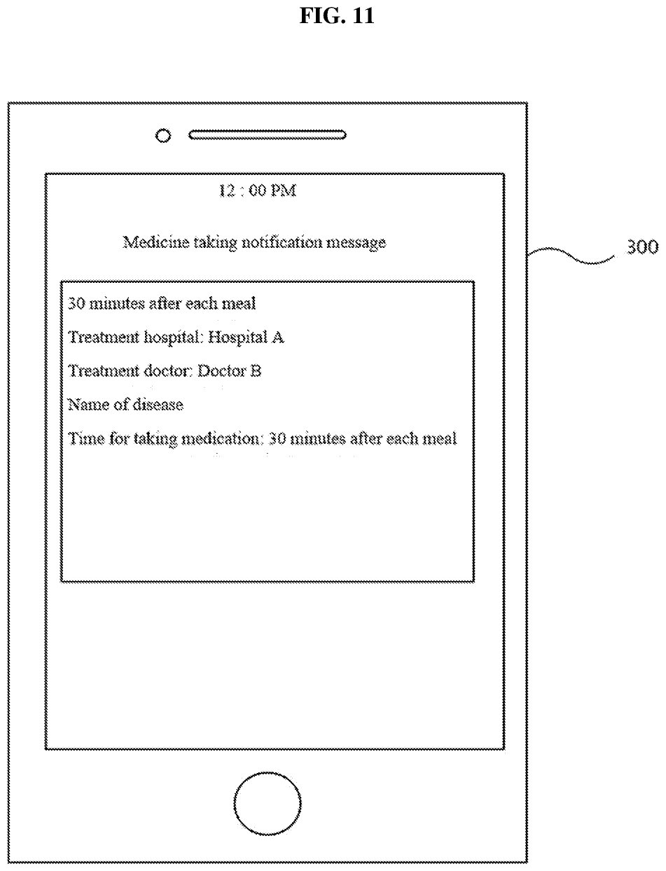

FIG. 11 is a drawing illustrating an example where a user application displays a medicine taking notification message according to another embodiment of the inventive concept.

Referring to FIG. 11, a user application 300 may receive medicine prescription information according to a treatment result from a medical staff client 500 and may store the medicine prescription information as medicine taking information in a storage unit of the user application 300.

When it is time to take a medication depending on medicine taking information stored in the storage unit, a notification unit of the user application 300 may display a medicine taking notification message on a display unit.

Preferably, the medicine taking notification message may include information about a time for taking a medicine, a treatment hospital, a treatment doctor, a name of disease, or the like.

The above-mentioned method for providing the healthcare data according to an embodiment of the inventive concept may be combined with a server which is hardware and may be stored in a medium to be implemented as a program (or application) to be executed.

For the computer to read the program and execute the methods implemented with the program, the above-mentioned program may include a code coded into a computer language such as C, C++, Java, or a machine language readable through a device interface of the computer by a processor (CPU) of the computer. Such a code may include a functional code associated with a function and the like defining functions necessary for executing the methods and may include a control code associated with an execution procedure necessary for the processor of the computer to execute the functions according to a procedure. Further, such a code may further include a code associated with memory reference about whether additional information or media necessary for the processor of the computer to execute the functions is referred at any location (address number) of an internal or external memory of the computer. Further, if it is necessary for the processor of the computer to communicate with any computer or server located in a remote place to execute the functions, the code may further include a communication related code about how communication is performed with any computer or server located in a remote place using a communication module of the computer and whether to transmit and receive any information or media upon communication.

The medium may refer to a device-readable medium which stores data on a semipermanent basis rather than a medium, such as a register, a cache, or a memory, which stores data during a short moment. The medium may refer to a device-readable medium which stores data on a semipermanent basis rather than a medium, such as a register, a cache, or a memory, which stores data during a short moment. In other words, the program may be stored in various storage media on various servers accessible by the computer or various storage media on the computer of the user. Further, the medium may be distributed to a computer system connected over a network and may store a computer-readable code on a distributed basis.

Operations of the method or algorithm described in connection with an embodiment of the inventive concept may be directly implemented in hardware, may be implemented with a software module executed by hardware, or may be implemented by a combination of the hardware and the software module. The software module may reside on a random access memory (RAM), a read only memory (ROM), an erasable programmable ROM (EPROM), an electrically erasable programmable ROM (EEPROM), a flash memory, a hard disc, a removable disc, a CD-ROM, or any type of computer-readable storage medium which is well known in the technical field to which the inventive concept pertains.

According to an embodiment of the inventive concept, the doctor may view health information and medical information of a patient and may treat the patient, thus making a quick, accurate diagnosis to the patient.

Furthermore, according to an embodiment of the inventive concept, the doctor checks a family history of a patient to treat the patient, thus making a more accurate diagnosis to the patient.

Furthermore, according to an embodiment of the inventive concept, the application may monitor whether there is an abnormal symptom of a child using information about oxygen saturation and a pulse of the child, thus immediately coping with an abnormal state which occurs in the child.

While the inventive concept has been described with reference to exemplary embodiments, it will be apparent to those skilled in the art that various changes and modifications may be made without departing from the spirit and scope of the inventive concept. Therefore, it should be understood that the above embodiments are not limiting, but illustrative.

What is claimed is:

1. A method for providing healthcare data, the method comprising:
receiving, by a server, a request to view personal health data of a specific user from a medical staff client, the personal health data including health information or medical information and being stored in a user application;
permitting, by the server, the medical staff client to view the personal health data as the user approves to view the personal health data; and
ending the permission to view the personal health data, as a certain time elapses or as medical staff store chart data,
wherein the health information includes at least one of body information, a family history, or a social history of the user,
wherein the medical information includes at least one of symptom information about a symptom of the user or information obtained through hospital treatment, and
wherein the chart data is stored by adding medical information depending on operations of the medical staff after the personal health data of the user is viewed by the medical staff client.

2. The method of claim 1, wherein the permitting to view the personal health data includes:
transmitting, by the server, a message requesting to view the personal health data to the user application; and
receiving, by the server, an approval time for viewing and a time limit for viewing from the user application and permitting, by the server, the medical staff client to view the personal health data, and
wherein the ending of the permission to view the personal health data includes:
ending the permission to view the personal health data as the time limit for viewing elapses or as the medical staff store the chart data.

3. The method of claim 1, wherein the permitting to view the personal health data includes:
receiving, by the server, the request to view the personal health data and a viewing authentication number from the medical staff client;
matching, by the server, the received viewing authentication number with a viewing authentication number stored in the user application; and
permitting, by the server, the medical staff client to view the personal health data, when the viewing authentication numbers are identical to each other according to the matched result.

4. The method of claim 1, further comprising:
transmitting, by the server, a message requesting to extend a viewing time to the user application, when receiving the message from the medical staff client; and
additionally permitting, by the server, the medical staff to view the personal health data by a time input from the user application.

5. The method of claim 1, further comprising:
receiving, by the server, the symptom information about the symptom of the user from the user application and receiving, by the server, a request to recommend a hospital according to the symptom information; and
analyzing, by the server, the symptom information to select a department and recommending, by the server, one or more hospitals according to the department to the user application.

6. The method of claim 1, further comprising:
inquiring, by the server, about a specialty of a specific medical staff client, when receiving a request to view personal health data of the user from the specific medical staff client;
determining, by the server, that the user is in an emergency state, when the specialty corresponds to an emergency department; and
permitting, by the server, the specific medical staff client to view the personal health data.

7. The method of claim 1, wherein the permitting to view the personal health data further includes:
permitting, by the server. the medical staff client to view personal health data of the family of the user or personal health data of a child of the user, as the user approves to view the personal health data,
wherein the personal health data of the family of the user is personal health data of a family member who is approved to share the personal health data with the user, and
wherein the personal health data of the child of the user is personal health data of a child who is registered with a user application account and includes measurement data being received from a sensor worn on a body of the child and being stored in the user application.

8. A server for providing healthcare data, the server comprising:
a reception unit configured to receive a request to view personal health data of a specific user from a medical staff client; and
a viewing permission unit configured to permit the medical staff client to view the personal health data as the user approves to view the personal health data,
wherein the personal health data includes health information or medical information and is stored in a user application,
wherein the health information includes at least one of body information, a family history, or a social history of a patient,
wherein the medical information includes at least one of symptom information about a symptom of the user or information obtained through hospital treatment, and
wherein the chart data is stored by adding medical information depending on operations of medical staff after the personal health data of the user is viewed by the medical staff client.

9. The server of claim 8, wherein the user application receives and stores personal health data including health information or medical information of a child of the user, receives and monitors measurement data for oxygen saturation and a pulse of the child of the user from a sensor worn or added to a body of the child, displays a warning message when an abnormal symptom of the child is discovered, and stores the measurement data and the abnormal symptom of the child in the personal health data of the child.

10. The server of claim 8, wherein the reception unit receives a request to view personal health data of a child of the specific user from the medical staff client, and
wherein the viewing permission unit permits the medical staff client to view the personal health data of the child as the user approves to view the personal health data and ends the viewing as a certain time elapses or as the medical staff store chart data.

11. A program for providing healthcare data, the program being combined with a computer which is hardware and being stored in a medium to execute the method of claim 1.
